(19) United States
(12) Patent Application Publication
(10) Pub. No.: US 2008/0292693 A1
(43) Pub. Date: Nov. 27, 2008

(54) COMPOSITION AND METHOD FOR TREATING DIGESTIVE SYSTEM DISORDERS

(76) Inventor: Scott F. Sones, Niland, CA (US)

Correspondence Address: JENNINGS, STROUSS & SALMON, P.L.C., 201 E. WASHINGTON ST., 11TH FLOOR, PHOENIX, AZ, 85004, US

(21) Appl. No.: 11/751,562

(22) Filed: May 21, 2007

(51) Int. Cl. A61K 36/752; A61K 9/48; A61P 1/00

(52) U.S. Cl. 424/456; 424/736

(57) ABSTRACT

The present disclosure provides for a novel composition, with active ingredients comprising d-Limonene and Aloe mucilaginous polysaccharides (AMPs), for the treatment of a wide variety of digestive system disorders and diseases, including conditions which affect the stomach, esophagus, bowel, colon, liver or pancreas are considered digestive system diseases and disorders, including, but not limited to, gastro-esophagus reflux disease (GERD), digestive problems such as acid reflux disease, Crohn's disease, colitis, inflammatory bowel disease (IBD), irritable bowel disease (IBS), diverticulitis, gastritis, peptic ulcers and spastic colon.

### FIELD OF THE INVENTION

This invention relates generally to the field of compositions and methods for treating digestive system disorders, and more specifically to a composition comprised of d-Limonene and Aloe mucilaginous polysaccharides (AMPs) and method of using the same for the treatment of a variety of digestive system disorders.

### BACKGROUND OF THE INVENTION

The gastrointestinal (GI) tract is a long hollow tube stretching from the head to the end of the body, including the mouth, pharynx, esophagus, stomach, small intestine, and large intestine. The salivary glands, liver, gallbladder, and pancreas are also important parts of this far-reaching system. The main purpose of the GI tract is to break down carbohydrates, fats, and proteins into molecules small enough to be absorbed through cell membranes. In that way, it provides the cells with the necessary energy for life and health.

Digestion begins in the mouth when food is chewed and starch is broken down by ptyalin, an enzyme secreted in saliva. Food then enters the stomach, where it is reduced to tiny particles and further transformed by gastric juices. The solid portion remains in the stomach for one to six hours until it liquefies completely; liquid passes into the small intestine, where numerous enzymes produced by the pancreas, along with bile from the liver, break it down further for absorption. When it finally arrives in the large intestine, all nutritional value has been spent, and the only remaining process is the removal of water before final elimination. Every section of the GI tract is prone to its own unique disorders—some merely annoying, others potentially fatal.

The most common of the digestive system diseases and disorders is heartburn. At least twenty-five percent of people in the United States and in other western countries experience heart burn once or more times per month, according to a study from the Mayo Clinic. In contrast, only eleven percent of those living in Eastern Asian countries have heartburn once or more per month. Excess stomach acid is what causes heartburn, but stress and anxiety are nervous disorders that can affect the digestive tract and increase stomach acid. Heartburn is a symptom of another of the common digestive system diseases and disorders: acid reflux.

Acid reflux or gastro-esophageal reflux disease (GERD) symptoms include heartburn, difficulty swallowing, regurgitation of stomach acid and/or food, hoarseness, coughing and irritation of the larynx, throat and/or esophagus. This is one of the digestive system diseases and disorders that can lead to more serious conditions if left untreated. For example, acid reflux or GERD is one of the major risk factors for esophageal cancer. As with heartburn, anxiety and stress are nervous disorders that affect the digestive tract and can aggravate acid reflux symptoms.

Diverticulitis occurs in ten to twenty-five percent of people with diverticulosis. Approximately half of all Americans age sixty to eighty, and almost everyone over age eighty, have diverticulosis at some time. The disease is common in developed or industrialized countries, particularly the United States, England, and Australia.

Ulcerative colitis is another of the digestive system diseases and disorders. This one can be very serious and when symptoms are severe may require surgery to remove diseased portions or the entire colon. Anxiety, stress, depression or emotional turmoil (all nervous disorders that can affect the digestive tract) are often experienced by people with ulcerative colitis and can worsen symptoms. It is an inflammatory bowel disease, similar to Crohn's disease. Whereas irritable bowel syndrome is not considered an inflammatory disease, but because the names are similar, people sometimes get them confused.

Irritable bowel syndrome or IBS is one of the common digestive system diseases and disorders. Somewhere between 25 and 55 million Americans suffer from IBS and most of them are women. IBS is not life threatening and it does not lead to other more serious digestive system diseases and disorders, but it can still be hard to live with. IBS and nervous disorders that affect the digestive tract, such as stress and anxiety, may cause a vicious circle in a person's life.

Therefore, a need exists for a composition and method for the treatment of a wide spectrum of digestive tract disorders.

### DETAILED DESCRIPTION OF VARIOUS EMBODIMENT

The present disclosure provides for a novel composition, method of administration and method of manufacture of a composition for the treatment of a wide variety of digestive system disorders and diseases. According to the present disclosure, conditions which affect the stomach, esophagus, bowel, colon, liver or pancreas are considered digestive system diseases and disorders, including, but not limited to, gastro-esophagus reflux disease (GERD), digestive problems such as acid reflux disease, Crohn's disease, colitis, inflammatory bowel disease (IBD), irritable bowel disease (IBS), diverticulitis, gastritis, peptic ulcers and spastic colon.

The active ingredients of the composition include d-Limonene and Aloe mucilaginous polysaccharides (AMPs). The disclosed combination may then be combined with any suitable pharmaceutical vehicle to provide the composition claimed herein. The composition is preferably administered once per day using two soft gel capsules or as a liquid. However, according to the present disclosure, methods of administration include tablet, capsule (hard, soft and gel caps), liquid, granulates, syrups and injectables. The composition is manufactured using conventional methods of pharmaceutical manufacture, including excipients, and by combining the d-Limonene and Aloe mucilaginous polysaccharides (AMPs) accordingly into one administrable composition.

The novelty of this invention rests in the combined use of d-Limonene and Aloe mucilaginous polysaccharides (AMPs). Preferably food grade d-Limonene with a purity of ninety-nine percent or greater is used. d-Limonene is the major component of the oil extracted from citrus rind. When citrus fruits are juiced, the oil is pressed out of the rind. This oil is separated from the juice, and distilled to recover certain flavor and fragrance compounds. The bulk of the oil is left behind and collected. This is food grade d-Limonene. d-Limonene is also available in synthetic form as dl-Limonene.

In two double-blind clinical studies, d-Limonene, administered as a daily dose of 1,000 mg, has been shown to significantly reduce the symptoms of GERD and Crohn's disease. In one GERD study, fifty percent of responders were free of GERD symptoms for up to six months without taking an additional dose of d-Limonene. Further, the Aloe-sourced AMP's ability to enhance specific nutrient bio-availability works harmoniously with d-Limonene to increase the bio-availability and actions of the d-Limonene compound.

Aloe mucilaginous polysaccharides are one of over two hundred nutrients found in and derived from Aloe vera plants. Specifically, AMPs are long chain sugars molecules composed of individual mannose and sugar molecular connected together. AMPs are the sole compound responsible for the healing properties attributed to Aloe vera. AMPs are normally categorized by chain size into four categories, depending on the number of molecules in the AMP chain.

According to the present disclosure, any or all categories of AMP molecules can be used, including, but not limited to: (1) small or 50-600 molecules; (2) medium or 601-1,500 molecules; (3) large or 1,501-5,000 molecules and (4) very large or greater than 5,000 molecules. To that end, in one embodiment, AMPs are provided as 200× full spectrum powder. However, it is preferable that a sufficient quantity of large or very large molecules are used, in that these categories tend to demonstrate higher efficacy in AMPs anti-inflammatory properties, protection against Colitis, anti-bacterial properties, improved immune system, reduced blood sugar levels, and anti-oxidant properties.

Unlike present treatments for digestive system disorders, such as esomeprazole (Nexium®), proton pump inhibitors (PPIs) and antacids, Aloe-sourced ingredients, such as AMPs have been clinically demonstrated to enhance specific nutrient bio-availability. Aloe-sourced ingredients, such as AMPs, also provide a natural time release mechanism for nutrients.

In one embodiment of the present invention, d-Limonene is present in a therapeutic amount ranging from 500 milligrams (mg) to 2,000 mg, with AMP being present in a therapeutic amount ranging from 800 mg to 4,000 mg. In an alternate embodiment, the composition comprises approximately 1,000 mg of d-Limonene and 1,000 mg of AMP.

The mechanisms of action of the disclosed composition in treating digestive system disorders include, but are not limited to, providing a barrier in the stomach and esophagus against bacterial infection, promoting quicker movement of food and gastric juices out of the stomach so that esophageal irritants do not promote as much reflux, and reducing the amounts of gastric juices that reflux back into the esophagus. Additionally, minor burping that occurs with ingestion of the compound may cause the composition to be carried directly into the esophagus, coating the esophagus against caustic contents that would have been regurgitated from the stomach.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s).

The foregoing description of a preferred embodiment, and best mode of the invention known to the applicant at this time of filing the application, have been presented and is intended for the purposes of illustration and description. It is not intended to be exhaustive nor limit the invention to the precise form disclosed and many modifications and variations are possible in the light of the above teachings. The embodiment was chosen and described in order to best explain the principles of the invention and its practical application and to enable others skilled in the art to best utilize the invention in various embodiments and with various modifications as are suited to the particular use contemplated. Therefore, it is intended that the invention not be limited to the particular embodiments disclosed for carrying out the invention.

1. A composition for treating digestive system disorders in humans, comprising: d-Limonene and Aloe mucilaginous polysaccharides.

2. The composition of claim 1, wherein the d-Limonene comprises synthetic dl-Limonene.

3. The composition of claim 1, wherein the d-Limonene is present in an amount in a range of approximately 500 mg to 2,000 mg.

4. The composition of claim 1, wherein the Aloe mucilaginous polysaccharides are present in an amount in a range of approximately 800 mg to 4,000 mg.

5. The composition of claim 1, wherein the d-Limonene is present in an approximate amount of 1,000 mg.

6. The composition of claim 1, wherein the Aloe mucilaginous polysaccharides is present in an approximate amount of 1,000 mg.

7. The composition of claim 1, wherein the Aloe mucilaginous polysaccharides comprise large Aloe mucilaginous polysaccharides.

8. The composition of claim 1, further including a pharmaceutically acceptable carrier.

9. The composition of claim 8, wherein the pharmaceutically acceptable carrier is selected from a group consisting of a hard capsule, a soft capsule, a gel capsule, a tablet, a liquid, granulates, a syrup and an injectable.

10. The composition of claim 1, wherein the d-Limonene comprises food grade d-Limonene.

11. The composition of claim 10, wherein the food grade d-Limonene is at least ninety-nine percent pure.

12. The composition of claim 1, wherein the composition is capable of reducing the symptoms of a digestive system disorder.

13. The composition of claim 12, wherein the digestive system disorder is gastro-esophagus reflux disease.

14-18. (canceled)

19. A method for manufacturing a composition for treating a digestive system disorder, comprising: combining d-Limonene and Aloe mucilaginous polysaccharides with a pharmaceutically acceptable carrier.

20. The method of claim 19, wherein the pharmaceutically acceptable carrier is selected from a group consisting of a hard capsule, a soft capsule, a gel capsule, a tablet, a liquid, granulates, a syrup and an injectable.
